(19) United States
(12) Patent Application Publication
(10) Pub. No.: US 2012/0078208 A1
(43) Pub. Date: Mar. 29, 2012

(54) Contained ostomy appliance

(76) Inventor: David A. Laudick, Spring City, TN (US)

(21) Appl. No.: 13/199,967

(22) Filed: Sep. 14, 2011

Related U.S. Application Data
(60) Provisional application No. 61/403,992, filed on Sep. 25, 2010.

(51) Int. Cl. A61F 5/445

(52) U.S. Cl. 604/332

(57) ABSTRACT

The present invention is an individual pressure barrier pouch that is used in conjunction with the assembly of either a one piece or a two piece ostomy appliance or as a pressure barrier pouch that can be preassembled and integrated into the assembly of a two piece ostomy appliance. In all three embodiments, body waste material/excretions from the patient's stoma/fistula pass through the pressure barrier pouch to enter the ostomy pouch. When pressure is applied to the ostomy pouch, the excretions no longer have access to escape through the path of least resistance between the stoma and the wafer stoma clearance hole as they do with a conventional ostomy pouch. The only access to the path of least resistance with the present invention would have body excretions backing up through the pressure barrier pouch.

### CROSS-REFERENCE TO RELATED APPLICATIONS

Provisional Patent Application: 61/403,992

### STATEMENT REGUARDING FEDERALLY SPONSORED RESEARCH OR DEVELOPMENT

Not Applicable

### BACKGROUND OF THE INVENTION

The present invention is in the technical field of ostomy appliance, more particularly, the present invention is in the technical field of an ostomy appliance improvement. The typical ostomy appliance is a system made up of a wafer and an ostomy pouch used to collect body waste/excretions and flagellant gas that is discharged from a patient's stoma/fistula. The ostomy appliance can be a one piece or a two piece system. With a one piece ostomy appliance, the wafer and the ostomy pouch are manufactured as one piece. The patient either cuts a hole in the wafer large enough to clear the stoma/fistula and allow passage of the excretions through the wafer into the ostomy pouch or purchases one with an appropriately sized wafer and clearance hole. The wafer is attached to the body with an adhesive backing. An optional barrier paste can also be used to help fill the void that exists between the wafer clearance hole and the patient's stoma/fistula. A two piece ostomy appliance consists of two independent pieces, the wafer and the ostomy pouch. The wafer in a two piece system attaches to the patients' body in the same way as the one piece appliance, but is manufactured with a flange that allows a snap fit with a mating flange on the ostomy pouch. The snap feature on a two piece ostomy appliance allows the ostomy pouch to be removed and replaced as needed without having to replace the wafer. This invention can be used as effectively with a one piece ostomy appliance or a two piece ostomy appliance.

With the current technology there is a high probability that an ostomy patient will incur a breach of body waste material/excretions or flagellant gas on numerous occasions. Ostomy products on the market today are not providing adequate protection against these breaches. This leakage commonly occurs between the skin and the adhesive backed wafer when pressure has been applied to the Ostomy pouch and is transferred directly to the seal around the stoma/fistula clearance hole in the wafer. The following attempts have been unsuccessful resolving these issues: U.S. Pat. No. 7,604,622 Issued Oct. 20, 2009 to Pedersen et al.; U.S. Pat. No. 7,789,866 Issued Sep. 7, 2010 to Poulsen; and Publication No. 20090234312 A1, Published Sep. 17, 2009 by O'Toole et al.

### SUMMARY OF THE INVENTION

The Contained Ostomy Appliance integrates a wafer and an ostomy pouch with a pressure barrier pouch. The pressure barrier pouch inhibits body waste material/excretions and flagellant gas that has been collected in the ostomy pouch from breaching the Contained Ostomy Appliance when pressure is applied to the ostomy pouch. It can be utilized with many different sizes and shapes of ostomy pouches and wafers available on the market today.

A first embodiment is a two piece appliance that illustrates an apparatus and assembly technique wherein the assembly of the pressure barrier pouch is integrated into the assembly of the ostomy pouch with a single attachment flange. This embodiment uses a body contact wafer with a single acting attachment flange to mate with the single attachment flange of the Contained Ostomy Appliance.

A second embodiment is also a two piece appliance that shows an apparatus and an assembly technique wherein the pressure barrier pouch is pre-assembled with its own attachment flange and then integrated during the assembly of the ostomy pouch. The ostomy pouch and the pressure barrier pouch each have their separate attachment flanges which snap onto a dual attachment flange on the body attachment wafer.

A third embodiment is a one piece appliance with an apparatus and an assembly technique wherein the assembly of a pressure barrier pouch is integrated in the assembly of an ostomy pouch that is fixedly attached to an adhesive backed body attachment wafer.

The advantages of the present invention without limitation will give ostomy patients confidence knowing they are protected from pressure related breaches, regardless whether the ostomy patient wears a one or two piece ostomy appliance, has a demanding occupation, works in construction, is a car mechanic, physically active, or works in a situation that requires moving in any unusual positions. The present invention, minimizes skin irritation around the stoma, extends the time between appliance changes, minimizes embarrassing breaches, and allows the patient to enjoy flexible eating times. The ostomy patient will be protected from pressure related breaches caused by clothes, belt lines, reaching, bending, tying shoes, and rolling in bed. The present invention lowers the anxiety that taking daily showers, sweating, and weight in the pouch will weaken the wafer adhesive bond inducing a pressure related breach.

### BRIEF DESCRIPTION OF THE DRAWINGS

FIG. 1 is a perspective view of an ostomy pouch shown in broken lines to illustrate the environment within which the need for a pressure barrier pouch exists and can be of the many shapes and sizes of ostomy pouches commercially available today.

FIG. 2 is an exploded perspective view of an ostomy pouch.

FIG. 3 is an exploded perspective view of a pressure barrier pouch.

FIG. 4 is an exploded perspective view of a first embodiment of a Contained two piece Ostomy Appliance.

FIG. 5 is a transparent front elevation view of thereof.

FIG. 6 is a transparent side view thereof showing the body attachment wafer, mating flange and sealable ostomy pouch drain in broken lines as they are not part of this invention.

FIG. 7 is a perspective view of an ostomy pouch shown in broken lines to illustrate environment of the second embodiment within which the need for a pressure barrier pouch exists.

FIG. 8 is an exploded view of a pressure barrier pouch thereof.

FIG. 9 is a perspective view of a pressure barrier pouch with attachment flange.

FIG. 10 is an exploded perspective view of a second embodiment of a Contained two piece Ostomy Appliance with separate attachment flanges.

FIG. 11 is a transparent front elevation view thereof.

FIG. 12 is a transparent side view thereof showing the body attachment wafer, mating double flange and sealable ostomy pouch drain in broken lines as they are not part of this invention.

FIG. 13 is an exploded perspective view of a third embodiment of a Contained one piece Ostomy Appliance.

FIG. 14 is a transparent front elevation view of thereof.

FIG. 15 is a transparent side view thereof showing the body attachment wafer and sealable ostomy pouch drain in broken lines as they are not part of this invention.

### DETAILED DESCRIPTION OF THE INVENTION

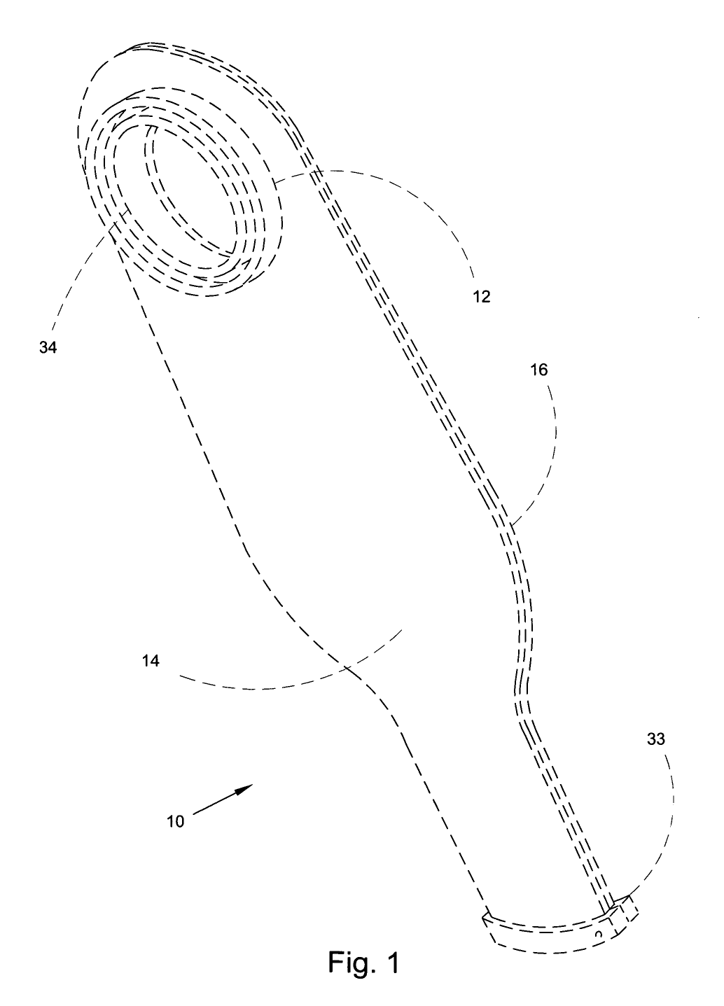

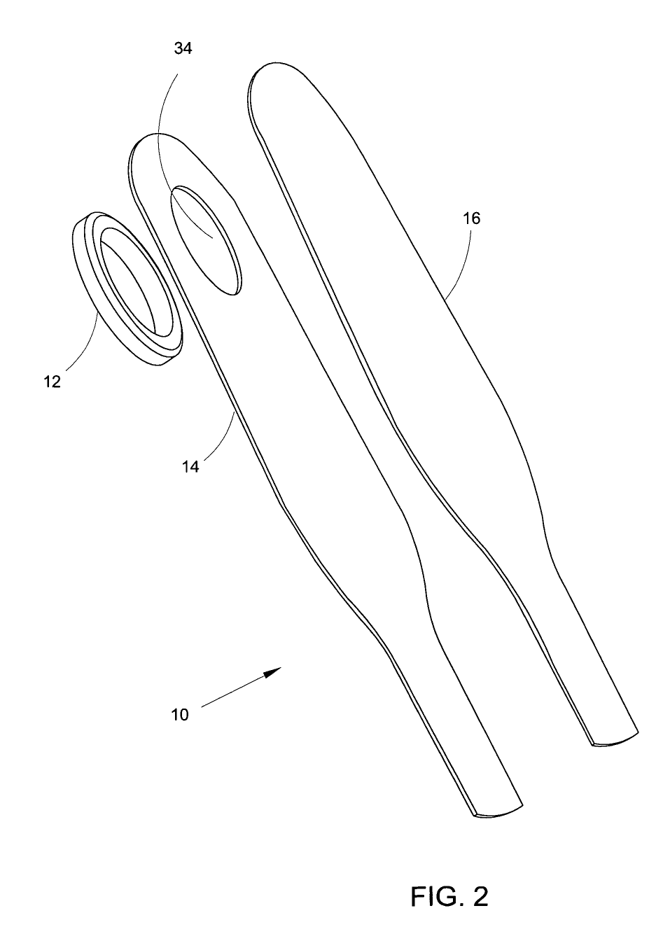

Similar parts will be referenced with common numerals throughout the several embodiments in the specification and the accompanying drawings. Ostomy pouch 10 is used to collect body waste/excretions and flagellant gas that is discharged from a patient's stoma/fistula. FIG. 1 and FIG. 2 show a perspective and an exploded view respectively of conventional ostomy pouch 10. Ostomy pouch 10 consists of attachment flange 12, second body or proximal side layer 14 with second stoma/fistula clearance opening 34, and second opposite or distal side layer 16. For drawing simplicity ostomy pouch 10 is shown constructed of two layers but is not limited to two layers. The construction detail is shown in FIG. 1 in broken lines to illustrate that ostomy pouch 10 can be manufactured in a wide variety of shapes and sizes to meet patient's needs. Ostomy pouch 10 is preferably constructed of plastic materials such as polyvinylchloride, or polyethylene, and manufactured with additional processes and features such as carbon filters, etc., that are not shown in the drawings or part of this invention. Assembly techniques used to manufacture ostomy pouch 10 are standard manufacturing processes such as stamping, heat staking, laser cutting, friction welding, sonic welding, crimping, and adhesives. The size, shape, materials, and manufacturing processes for ostomy pouch 10 are not limited to those mentioned here.

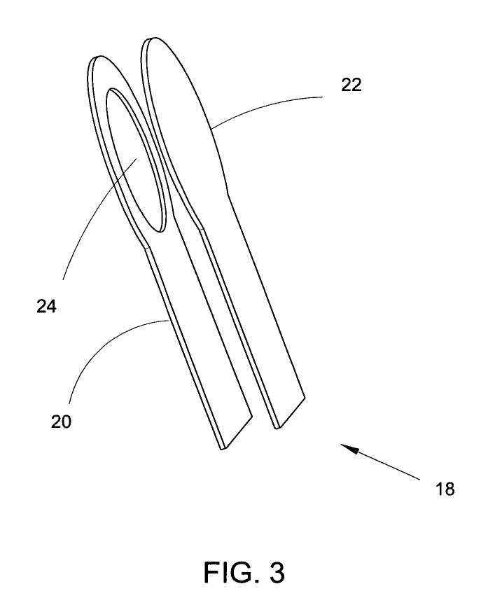

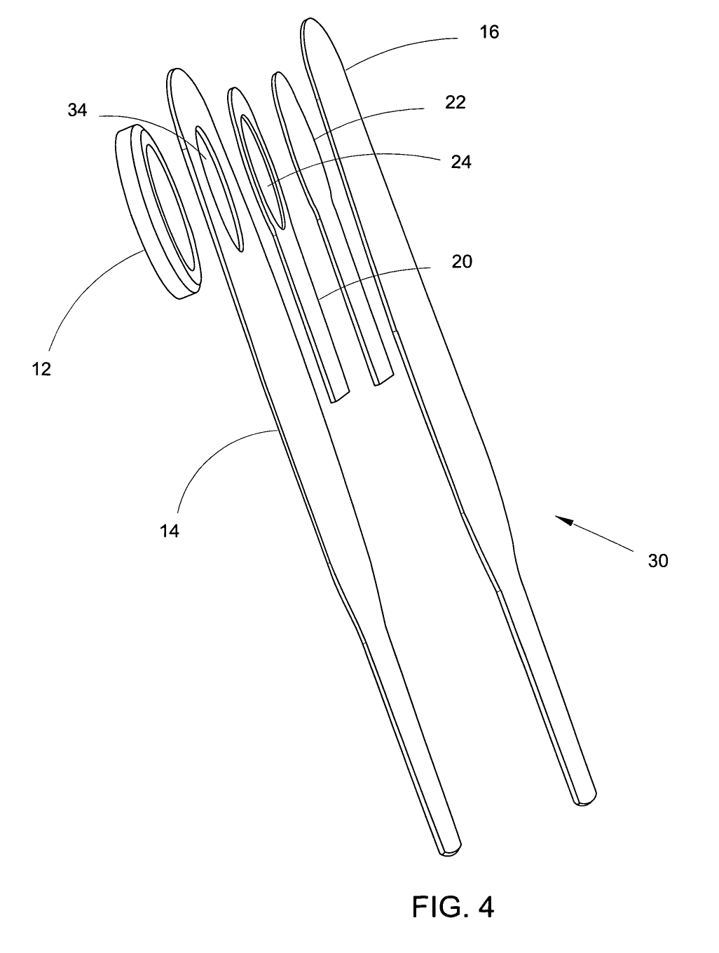

In a first embodiment shown in FIG. 3, in an exploded perspective view, pressure barrier pouch 18 assembly is shown and consists of first body or proximal side layer 20, and first opposite or distal side layer 22. In FIG. 4, pressure barrier pouch 18 is shown as an integral part of the assembly of Contained Ostomy Appliance 30. Pressure barrier pouch 18 inhibits body waste material/excretions and flagellant gas that has been collected in the ostomy pouch 10 from breaching Contained Ostomy Appliance 30 when the body waste materials/excretions and flagellant gas in ostomy pouch 10 are under pressure by providing a barrier to the path of least resistance to the seal between wafer 26 and the patient's body. Pressure barrier pouch 18 is shown made of two layers for drawing simplicity, but is not limited to two layers.

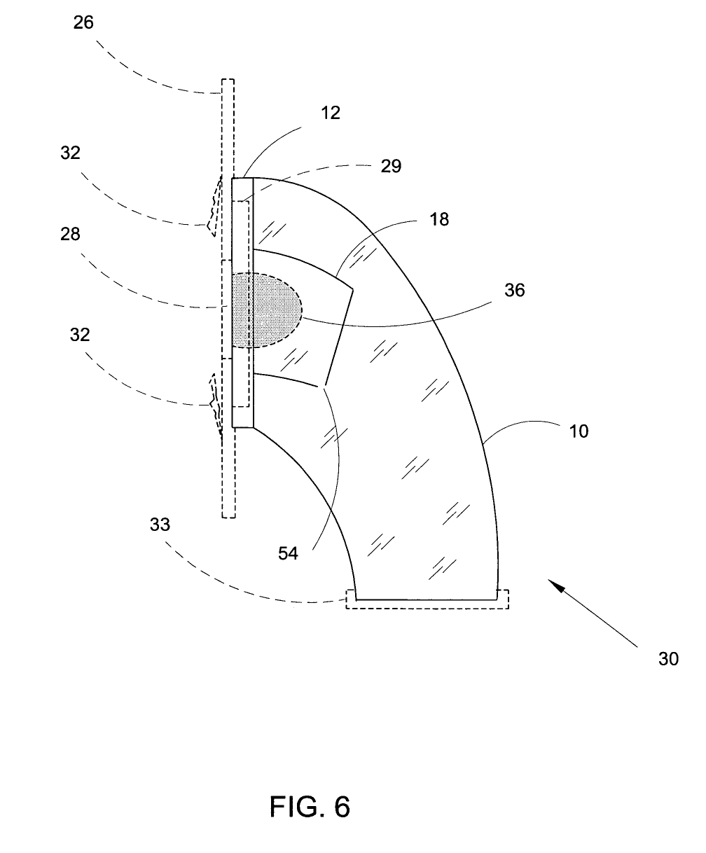

In further detail, pressure barrier pouch first proximal side layer 20 has first stoma/fistula clearance opening 24 that allows body waste/excretions from the patient's fistula/stoma 36 to pass into pressure barrier pouch 18 and out through opening 54 in the bottom of the pressure barrier pouch 18 into ostomy pouch 10 as shown in FIG. 6.

The construction and dimension details of Contained Ostomy Appliance 30 as shown in FIG. 3-6 are such that the overall size of pressure barrier pouch 18 is dependent on the size of ostomy pouch 10 that it mates with. For optimal performance in inhibiting pressure transfer to adhesive bond between wafer and patient's body, pressure barrier pouch 18 should be approximately 25% of the size of ostomy pouch 10, and preferably would be constructed of the same shapes, and plastic materials, polyvinylchloride or polyethylene, as ostomy pouch 10 that it is mated to. Assembly techniques used to manufacture pressure barrier pouch 18 are standard manufacturing processes such as stamping, laser cutting, heat staking, friction welding, sonic welding, crimping, and adhesives. The size, shape, features, materials and manufacturing processes for pressure barrier pouch 18 are not limited to those mentioned here.

An exploded perspective view of Contained Ostomy Appliance 30 is shown in FIG. 4 with attachment flange 12, second proximal side layer 14 of ostomy pouch 10 with second stoma/fistula clearance opening 34, first proximal side layer 20 of pressure barrier pouch 18 with first stoma/fistula clearance opening 24, first distal side layer 22 of pressure barrier pouch 18 and second distal side layer 16 of ostomy pouch 10.

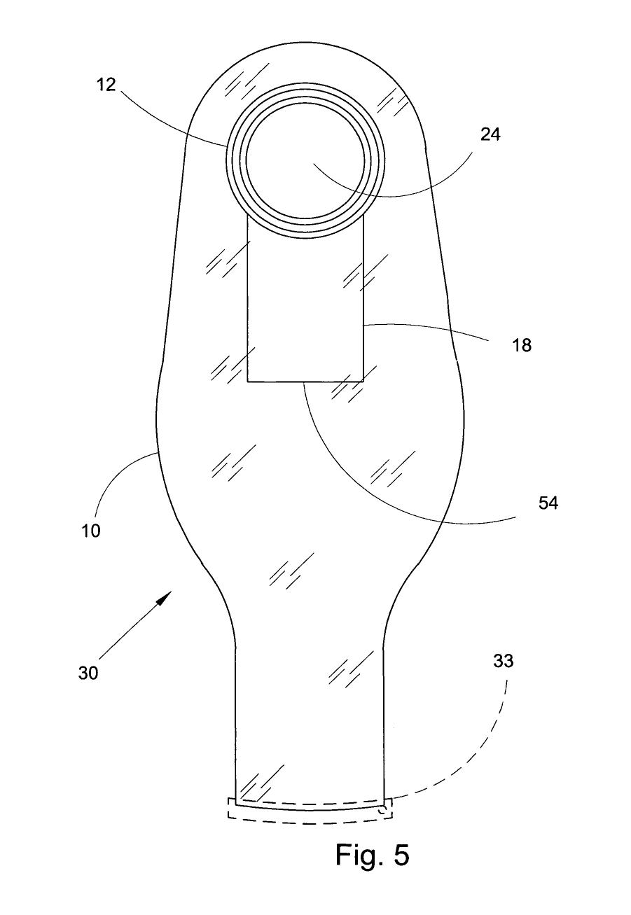

FIG. 5 and FIG. 6 show front and side transparent views respectively of Contained Ostomy Appliance 30 that after assembly consists of pressure barrier pouch 18 within ostomy pouch 10, connected to attachment flange 12.

Adhesive coated body attachment wafer 26, stoma/fistula clearance opening 28, attachment flange 29, barrier paste 32 and sealable ostomy pouch drain 33 are shown in broken lines for illustration purposes only in FIG. 6 and are not part of this invention. Stoma/fistula clearance opening 28 through attachment wafer 26 is placed over patient's stoma/fistula 36 and attached with an adhesive backing to the patients' body. Optional barrier paste 32 can also be used to help fill the void that exists between stoma/fistula clearance opening 28 in wafer 26 and patient's stoma/fistula 36.

Contained Ostomy Appliance 30 shown in FIGS. 1-6 is a two piece ostomy appliance wherein body attachment wafer 26 has attachment flange 29 that mates with attachment flange 12.

The first embodiment describes Contained Ostomy Appliance 30 wherein pressure barrier pouch 18 is assembled as part of the assembly process of an ostomy pouch 10 as shown in FIGS. 1-6. The assembly of the first embodiment is described in the three steps below. Step 1 indicates stamping/laser cutting of second proximal side layer 14 and second distal side layer 16 of ostomy pouch 10 to the same size and shape. Second stoma/fistula clearance opening 34 is cut in the top center of second proximal side layer 14. Integration of pressure barrier pouch 18 is preferably achieved by stamping/laser cutting pressure barrier pouch first proximal side layer 20 and first distal side layer 22 from plastic material to the same size and shape such that it comprises pressure barrier pouch 18 that is approximately 25% the size of mating ostomy pouch 10. Pressure barrier pouch 18 may be similar in shape to ostomy pouch 10 with the top being circular in shape then dropping straight down approximately ¾ of the way around the circular diameter towards the bottom and cut off after the appropriate length is achieved. After first distal side layer 22 and first proximal side layer 20 of pressure barrier pouch 18 are cut to size and shape, first proximal side layer 20 of pressure barrier pouch 18 requires first stoma/fistula clearance opening 24 to be positioned in the center of the top diameter and cut to approximately the same size as second stoma/fistula clearance opening 34 in second proximal side layer 14 of mating ostomy pouch 10.

Step 2 assembles first proximal side layer 20 and first distal side layer 22 of pressure barrier pouch 18 to second proximal side layer 14 of ostomy pouch 10. For simplicity, we will describe the assembly process using individual layers. Flange 12 of ostomy pouch 10 is positioned in a fixture with the snap side down. Second proximal side layer 14 of ostomy pouch 10 is then positioned and fixtured with second stoma/fistula clearance opening 34 centered over flange 12. First proximal side layer 20 of pressure barrier pouch 18 is then positioned and fixtured with first stoma/fistula clearance opening 24 centered over second stoma/fistula clearance opening 34 in second proximal side layer 14 of ostomy pouch 10 and with flange 12. Flange 12, second proximal side layer 14 of ostomy pouch 10 and first proximal side layer 20 of pressure barrier pouch 18 are then married together by standard manufacturing operations such as crimping, heat staking, etc.

Finally, in step 3 the final assembly process consists of attaching first distal side layer 22 of pressure barrier pouch 18 and second distal side layer 16 of ostomy pouch 10 to the sub-assembly completed in step 2. With the sub-assembly in step 2 fixtured with flange 12 down, first distal side layer 22 of pressure barrier pouch 18 is centered over pressure barrier pouch first proximal side 20 and is attached around the perimeter with a heat stake, adhesive, sonic weld or other manufacturing process. First distal side layer 22 is not sealed to proximal side layer 20 at the bottom of pressure barrier pouch 18 thus providing opening 54 to allow escape of body wastes into ostomy pouch 10. Second distal side layer 16 of ostomy pouch 10 is then centered and fixtured over second proximal side layer 14 of ostomy pouch 10 and attached through a heat stake, adhesive, sonic weld or other manufacturing process completely around the perimeter of ostomy pouch 10 except for the bottom of ostomy pouch 10 wherein a sealable drain 33 completes the assembly of Contained Ostomy Appliance 30.

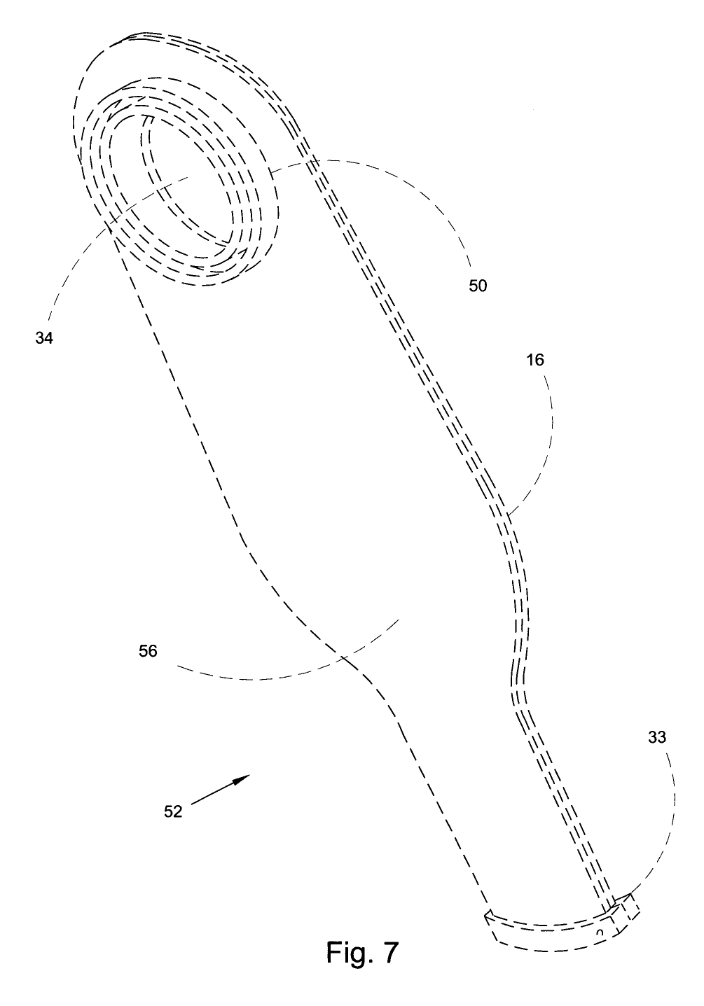

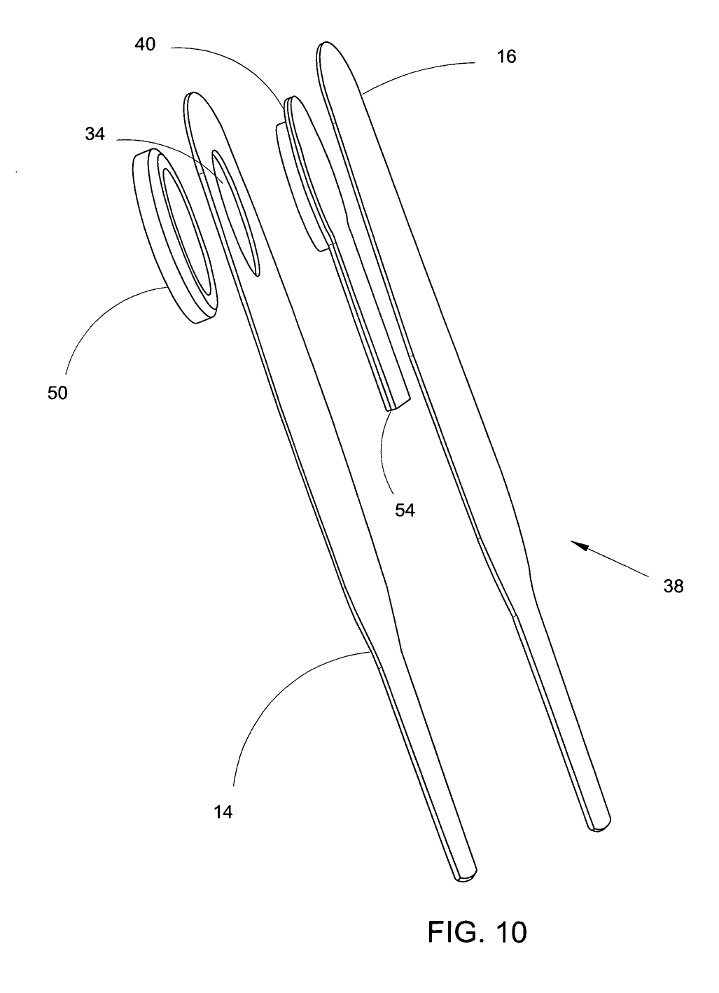

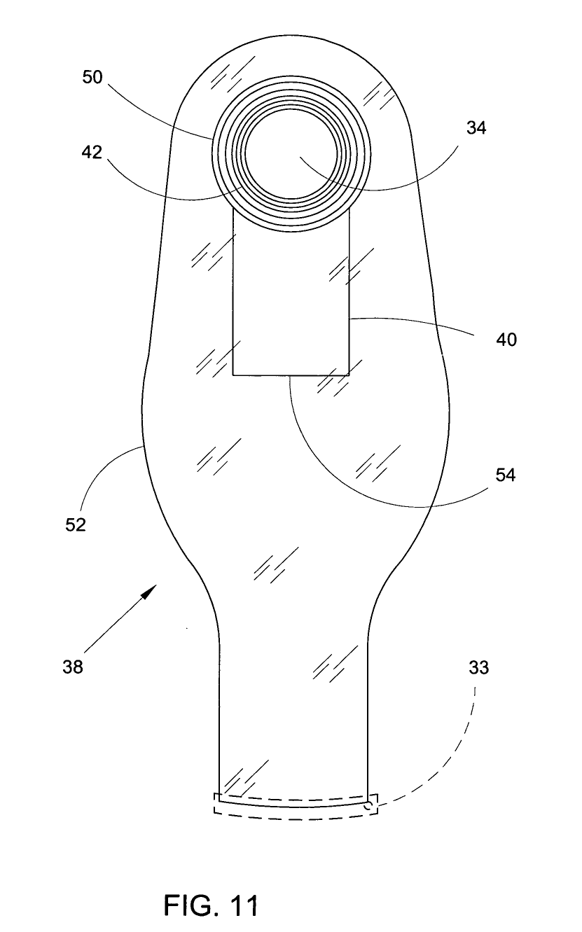

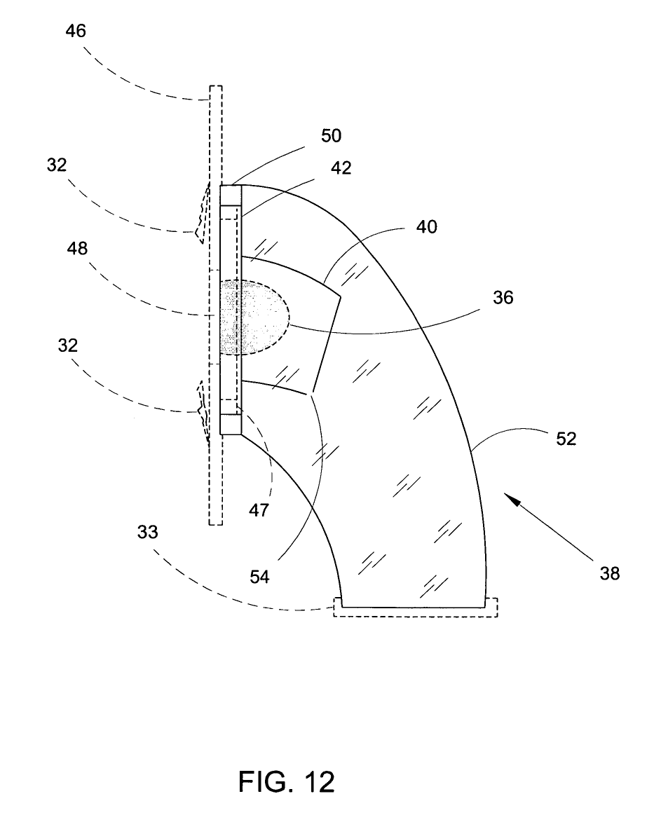

A second embodiment is shown in FIGS. 7-12 and shows pressure barrier pouch 40 that may be manufactured and sold separately to manufacturers that wish to include pressure barrier pouch 40 in their Contained Ostomy Appliances 38. Contained Ostomy Appliance 38 shown in FIGS. 7-12 is also a two piece ostomy appliance wherein body attachment wafer 46 has a dual snap feature that allows both pressure barrier pouch flange 42 and ostomy pouch flange 50 to connect to body attachment wafer 46. Adhesive coated body attachment wafer 46, stoma/fistula clearance opening 48, optional barrier paste 32 and sealable drain 33 are shown in broken lines for illustration purposes only in FIG. 12 and are not part of this invention. Stoma/fistula clearance opening 48 through attachment wafer 46 is placed over patient's stoma/fistula 36 and attached with an adhesive backing to the patients' body. Optional barrier paste 32 can also be used to help fill the void that exists between stoma/fistula clearance opening 48 in wafer 46 and patient's stoma/fistula 36.

FIG. 7 shows ostomy pouch 52 with second stoma/fistula clearance opening 34 and attachment flange 50 and second proximal side layer 56 and second distal side layer 16. Ostomy pouch 52 is used to collect body waste/excretions and flagellant gases that are discharged from the patient's stoma/fistula 36. Ostomy pouch 52 is shown in broken lines indicating that any of the conventional sizes and shapes of ostomy pouches may be used with this invention.

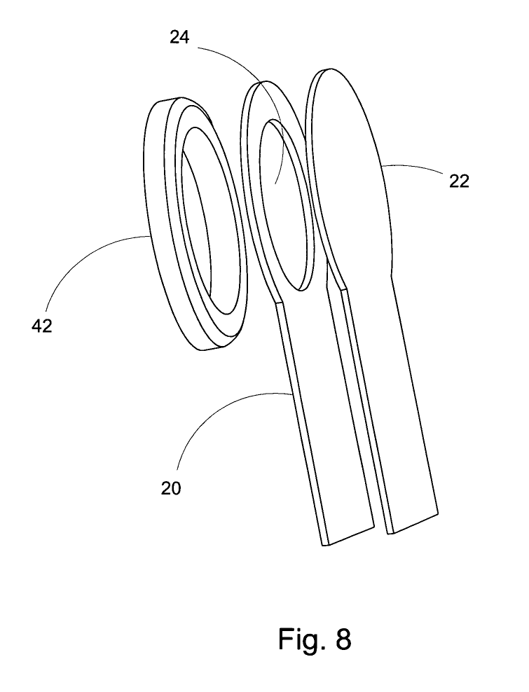

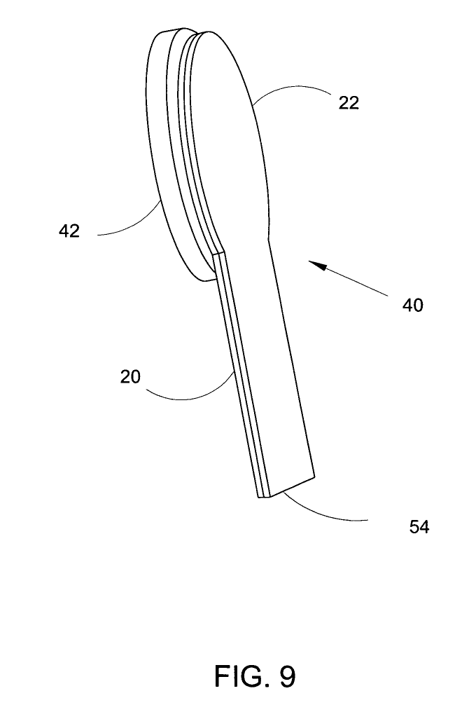

Referring now to FIG. 8 and FIG. 9, exploded and an assembled perspective views respectively, there is shown pressure barrier pouch 40 consisting of first proximal side layer 20, and first distal side layer 22 and attachment flange 42. Pressure barrier pouch 40 inhibits body waste material and flagellant gas that has been collected in ostomy pouch 52 from breaching outside Contained Ostomy Appliance 38 when the waste/excretions in ostomy pouch 52 are under pressure by providing a barrier to the path of least resistance to the seal between wafer 46 and the patient's body. Pressure barrier pouch 40 is shown made of two layers for drawing simplicity, but is not limited to two layers.

FIG. 8 and FIG. 12 shows pressure barrier pouch first proximal side layer 20 has first stoma/fistula clearance opening 24 that allows body waste/excretions and flagellant gas from the patient's fistula/stoma 36 to pass into pressure barrier pouch 40 and out through opening 54 in the bottom of pressure barrier pouch 40 into ostomy pouch 52.

The construction and dimension details of the invention as shown in FIGS. 7-12 are such that the overall size of pressure barrier pouch 40 is dependent on the size of conventional ostomy pouch 52 that it mates with. For optimum performance in inhibiting pressure transfer to adhesive bond between wafer and patient's body, pressure barrier pouch 40 is approximately 25% of the size of ostomy pouch 52, and would preferably be constructed of the same shapes, and plastic materials such as polyvinylchloride or polyethylene. Assembly techniques used to manufacture pressure barrier pouch 40 are standard manufacturing processes such as stamping, laser cutting, heat staking, friction welding, sonic welding, crimping, and adhesives. The size, shape, features, materials and manufacturing processes for pressure barrier pouch 40 are not limited to those mentioned here.

FIG. 12 shows a transparent side view of Contained Ostomy Appliance 38 comprised of pressure barrier pouch 40 and ostomy pouch 52, individually attached to double flange wafer 46. Mounting flange 42 of pressure barrier pouch 40 is shown fitting into mounting flange 50 of ostomy pouch 52.

In further detail, still referring to FIG. 12, double flange wafer 46 is applied over patient's stoma/fistula 36 through stoma/fistula clearance opening 48 in double flange wafer 46 and attached with an adhesive backing. Optional barrier paste 32 can also be used to help fill the void that exists between stoma/fistula clearance opening 48 in double flange wafer 46 and patient's stoma/fistula 36. Attachment of both pressure barrier pouch 40 and ostomy pouch 52 is completed by snapping flange 42 of pressure barrier pouch 40 and flange 50 of conventional ostomy pouch 52 to mating dual acting flange 47 mounted on double flange wafer 46. Double flange wafer 46, dual acting flange 47, stoma/fistula clearance opening 48, barrier paste 32 and sealable drain 33 are not part of this invention and are shown in broken lines for illustrative purposes only.

The assembly technique for this embodiment is described in the three steps below and shown in FIG. 9 to FIG. 12. Step 1 cuts pressure barrier pouch first proximal side layer 20 and first distal side layer 22 from plastic material to the same size and shape. Pressure barrier pouch 40 is similar in shape to ostomy pouch 52 with the top being circular in shape then dropping straight down approximately ¾ of the way around the circular diameter towards the bottom and cut off after the appropriate length is achieved. For optimum performance in inhibiting pressure transfer to adhesive bond between wafer and patient's body, pressure barrier pouch 40 is approximately 25% of the size of ostomy pouch 52. After first distal side layer 22 and first proximal side layer 20 of pressure barrier pouch 40 are cut to size and shape, first proximal side layer 20 of pressure barrier pouch 40 requires first stoma/fistula clearance opening 24 cut approximately to the same size as second stoma/fistula clearance opening 34 in the mating ostomy pouch 52. First stoma/fistula clearance opening 24 is centered on the top circular diameter of first proximal side layer 20 of pressure barrier pouch 40.

Step 2 assembles first proximal side layer 20 of pressure barrier pouch 40 to flange 42. Flange 42 is positioned face down in the fixture. First proximal side layer 20 of pressure barrier pouch 40 is positioned with first stoma/fistula clearance opening 24 centered over flange 42. First proximal side layer 20 of pressure barrier pouch 40 and flange 42 are then married together by a manufacturing operation such as crimping, heat staking, etc.

Step 3 consists of attaching first distal side layer 22 of pressure barrier pouch 40 to sub-assembly that was completed in step 2 above. Flange 42 of the sub-assembly is positioned face down in the fixture; first distal side layer 22 of pressure barrier pouch 40 is centered over first proximal side layer 20 of pressure barrier pouch 40 and attached around the perimeter except for the bottom with a heat stake, adhesive, sonic weld or other manufacturing process. First distal side layer 22 is not sealed to first proximal side layer 20 at the bottom of pressure barrier pouch 40, creating opening 54 to allow body wastes/excretions and flagellant gas to flow from pressure barrier pouch 40 to ostomy pouch 52. This completes the assembly of pressure barrier pouch 40 which can be integrated with any of the standard ostomy pouch 52 shapes and sizes by slipping the outside diameter of flange 42 into the inside diameter of flange 50 and sealing the joint between them during the assembly process of the ostomy pouch 52 selected.

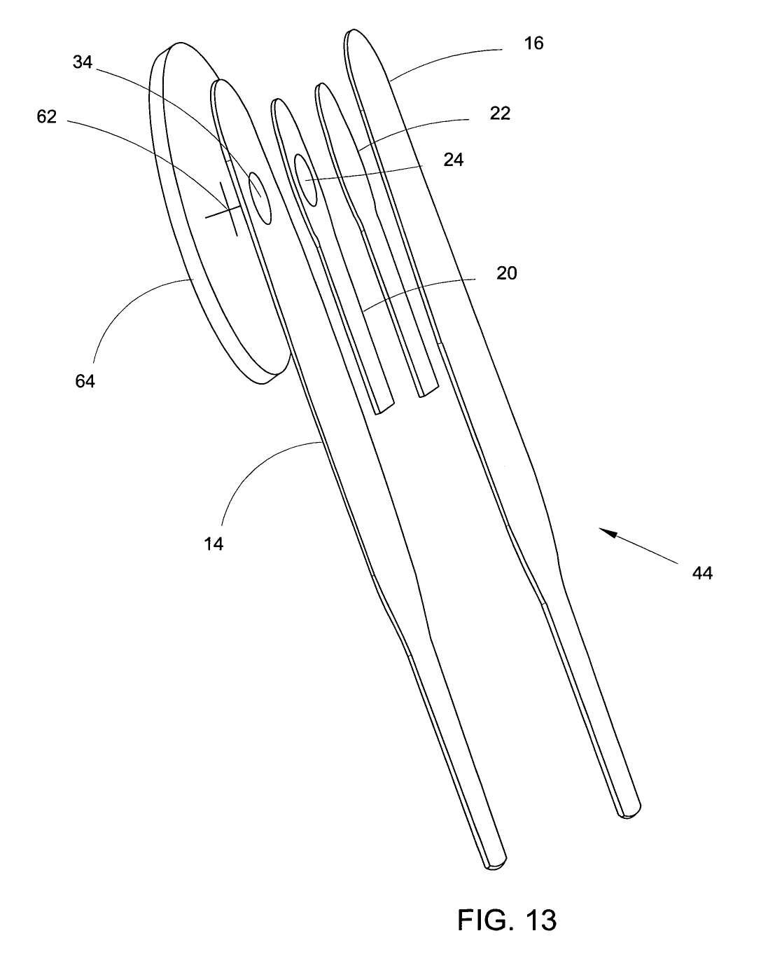

A third embodiment is shown in FIGS. 13-15 that describes pressure barrier pouch 60 integrated in Contained Ostomy Appliance 44 as a one piece assembly. Contained Ostomy Appliance 44 is comprised of wafer 64 being fixedly attached to ostomy pouch 58 with pressure barrier pouch 60 assembled internally in a similar manner to Contained Ostomy Appliances 30 and 38. A one piece installation of this inventive concept is shown in FIGS. 13-15. With this embodiment changing ostomy appliance 44 requires breaking the adhesive bond to the skin and reapplying adhesive when the replacement appliance 44 is ready. Contained Ostomy Appliance 44 has no attachment flange as its body attachment wafer 64 is fixedly attached to ostomy pouch 58 and pressure barrier pouch 60.

FIG. 13 shows body attachment wafer 64 with stoma/fistula clearance opening starter 62, ostomy pouch second proximal side layer 14 with second stoma/fistula clearance opening 34, pressure barrier pouch first proximal side layer 20 with first stoma/fistula clearance opening 24, pressure barrier pouch first distal side layer side 22 and ostomy pouch second distal side layer 16. Ostomy pouch 58 is used to collect body waste/excretions and flagellant gas that is discharged from the patient's stoma/fistula 36.

Pressure barrier pouch 60 inhibits body waste material/excretions and flagellant gas that has been collected in ostomy pouch 58 from breaching outside Contained Ostomy Appliance 44 when the waste/excretions and flagellant gases in ostomy pouch 58 are under pressure by providing a barrier to the path of least resistance to the seal between wafer 64 and the patient's body. Pressure barrier pouch 60 is shown made of two layers for drawing simplicity, but is not limited to two layers.

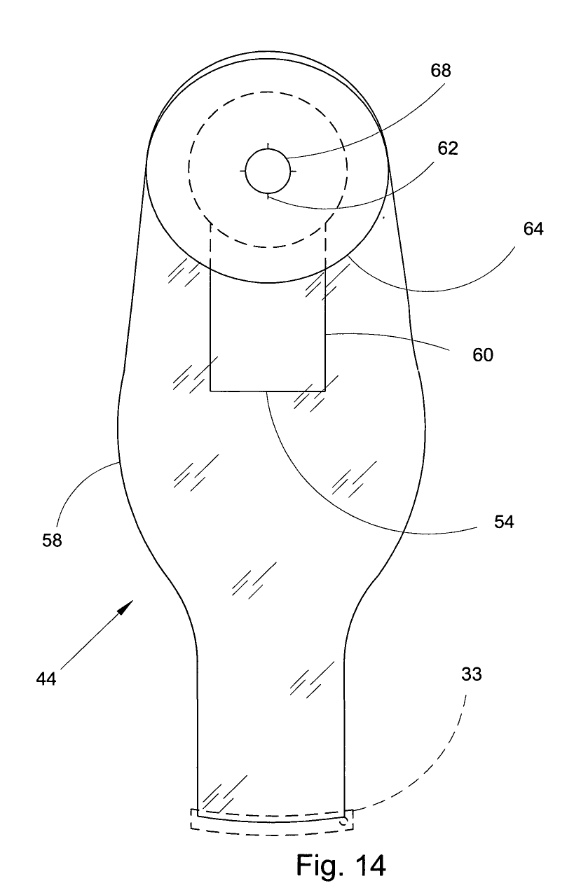

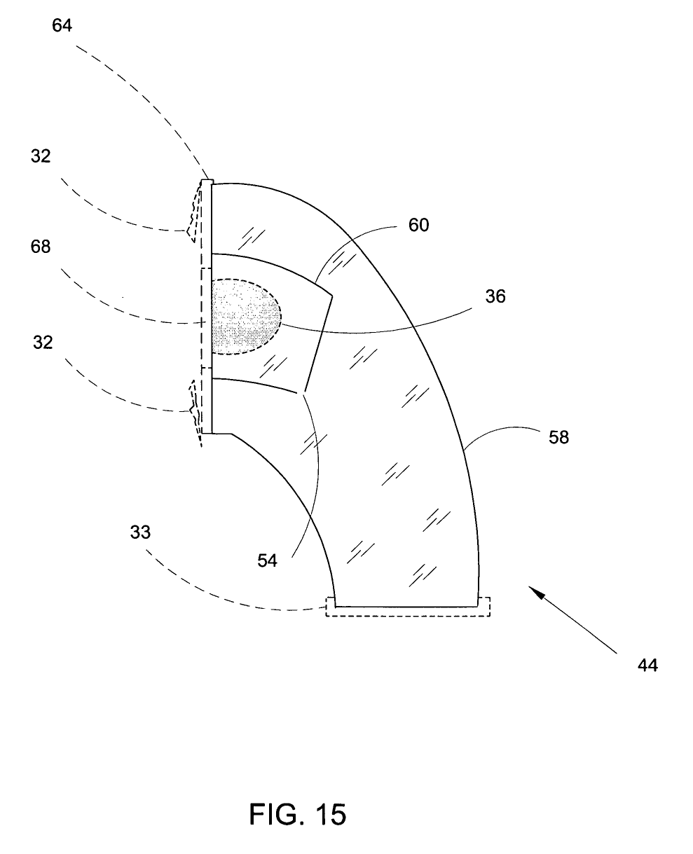

FIG. 14 and FIG. 15 shows stoma/fistula clearance opening starter 62 in wafer 64, stoma/fistula clearance opening 34 in second proximal side layer of ostomy pouch 14 and stoma/fistula clearance opening 24 in pressure barrier pouch first proximal side layer 20 respectively. Patient or caregiver either measures the size of the stoma/fistula 36 and using opening starter 62, cuts stoma/fistula clearance opening 68 through wafer 64 to allow body waste/excretions and flagellant gas from the patient's fistula/stoma 36 to pass into pressure barrier pouch 60 and out through opening 54 in the bottom of pressure barrier pouch 60 into ostomy pouch 58 or purchases attachment wafers with predetermined diameter stoma/fistula clearance openings.

The construction and dimension details of the invention as shown in FIGS. 13-15 are such that the overall size of pressure barrier pouch 60 is dependent on the size of conventional ostomy pouch 58 that it mates with. For optimum performance in inhibiting pressure transfer to adhesive bond between wafer and patient's body, pressure barrier pouch 60 is approximately 25% of the size of ostomy pouch 58, and preferably be constructed of the same shapes, and plastic materials such as polyvinylchloride or polyethylene. Assembly techniques used to manufacture pressure barrier pouch 60 and ostomy pouch 58 are standard manufacturing processes such as stamping, laser cutting, heat staking, friction welding, sonic welding, crimping, and adhesives. The size, shape, features, materials and manufacturing processes for pressure barrier pouch 60 are not limited to those mentioned here.

FIG. 15 shows a transparent side view of Contained Ostomy Appliance 44. FIG. 15 shows Contained Ostomy Appliance 44 comprised of pressure barrier pouch 60 and ostomy pouch 58, fixedly attached to body attachment wafer 64.

In further detail, after wafer 64 has its stoma/fistula clearance opening 68 enlarged to clear stoma/fistula 36, wafer 64 is attached to patient's body with an adhesive backing. Optional barrier paste 32 can also be used to help fill the void that exists between stoma/fistula clearance opening 68 in wafer 64 and patient's stoma/fistula 36.

The assembly technique for this embodiment is described in the three steps below and shown in FIGS. 13-15. Step 1 cuts pressure barrier pouch first proximal side layer 20 and first distal side layer 22 from plastic material to the same size and shape. Pressure barrier pouch 60 is similar in shape to ostomy pouch 58 with the top being circular in shape then dropping straight down approximately ¾ of the way around the circular diameter towards the bottom and cut off after the appropriate length is achieved. For optimum performance in inhibiting pressure transfer to adhesive bond between wafer and patient's body, pressure barrier pouch 60 is approximately 25% of the size of ostomy pouch 58. After first distal side layer 22 and first proximal side layer 20 of pressure barrier pouch 60 are cut to size and shape, first proximal side layer 20 of pressure barrier pouch 60 requires first stoma/fistula clearance opening 24 cut approximately on the same center as second stoma/fistula clearance opening 34 in the mating ostomy pouch second proximal side layer 14. Second stoma/fistula clearance opening 34 is centered on the top circular diameter of first proximal side layer 20 of pressure barrier pouch 60.

Step 2 assembles second proximal side layer 14 of ostomy pouch and first proximal side layer 20 of pressure barrier pouch 60 to wafer 64. Wafer 64 is positioned proximal side down in a fixture. Second proximal side layer 14 of ostomy pouch with second stoma/fistula clearance opening 34 is centered over wafer 64 aligning with stoma/fistula clearance opening starter 62. Second proximal side layer of ostomy pouch 14 is fixedly attached to wafer 64. First proximal side layer of pressure barrier pouch 20 is then centered in fixture aligning stoma/fistula clearance opening 24 with stoma/fistula clearance opening starter 62 and fixedly attaching wafer 64 and second proximal side layer of ostomy pouch 14 in the area of the wafer 64.

Step 3 consists of attaching first distal side layer 22 of pressure barrier pouch 60 to sub-assembly that was completed in step 2 above. Wafer 64 of the sub-assembly is positioned proximal side down in the fixture and first distal side layer 22 of pressure barrier pouch 60 is centered over first proximal side layer 20 of pressure barrier pouch 60 and attached around the perimeter with a heat stake, adhesive, sonic weld or other manufacturing process except first distal side layer 22 is not sealed to first proximal side layer 20 at the bottom of pressure barrier pouch 60, creating opening 54 to allow bodily wastes to flow from pressure barrier pouch 60 to ostomy pouch 58. Second distal side layer 16 of ostomy pouch 10 is then centered and fixtured over second proximal side layer 14 of ostomy pouch 10 and attached through a heat stake, adhesive, sonic weld or other manufacturing process completely around the perimeter of ostomy pouch 10 except for the bottom of ostomy pouch 10 wherein sealable drain 33 completes the assembly of the Contained Ostomy Appliance 44. Finally stoma/fistula clearance opening 68 is cut though attachment wafer 64 large enough to clear stoma/fistula 36 or is purchased with the stoma/fistula clearance opening 68 in attachment wafer cut to predetermined diameters. Again, this invention can be integrated with any of the standard ostomy pouch shapes and sizes.

While the foregoing written descriptions of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiments, methods, and examples, but by all embodiments and methods within the scope and spirit of the invention.

1. A contained ostomy appliance consisting of an improvement to an ostomy pouch for collecting body wastes/excretions and flagellant gas from a stoma/fistula comprising the integration of:
a pressure barrier pouch constructed with a minimum of a first proximal side layer and a first distal side layer wherein said first layers have a perimeter consisting of a top, sides and a bottom, with said first proximal side layer having a first stoma/fistula clearance opening large enough to clear said stoma/fistula, located toward said top of said first proximal side layer; wherein said perimeters of said first layers are attached to each other around said perimeter except for said bottoms that are left unattached, whereby said body wastes/excretions and flagellant gas are allowed to pass into
an ostomy pouch constructed with a minimum of a second proximal side layer and a second distal side layer wherein said second layers have a perimeter with a top and bottom, with said second proximal side layer having a second stoma/fistula clearance opening large enough clear said stoma/fistula, located toward said top of said second proximal side layer, aligned with said first stoma/fistula clearance opening in said first proximal side layer of said pressure barrier pouch wherein the areas surrounding said first and second stoma/fistula clearance openings are attached to each other;
said perimeters of said second layers are attached to each other except at said bottom of ostomy pouch wherein sealable drain completes said attachment, whereby pressure to outside of said ostomy pouch is not transferred directly to said stoma/fistula clearance openings in said first and second proximal side layers and breaches to said contained ostomy appliance are dramatically reduced or eliminated in all but the case where both of said pouches are completely full.

2. A contained ostomy appliance consisting of an improvement to an ostomy pouch for collecting body wastes/excretions and flagellant gas from a stoma/fistula as in claim 1 wherein said ostomy appliance is a one piece appliance with an adhesive backed attachment wafer fixedly mounted to said ostomy pouch and pressure barrier pouch with a stoma/fistula clearance opening starter that aligns with stoma/fistula clearance openings in said second proximal side layer of said ostomy pouch and first proximal side layer of said pressure barrier pouch, wherein a stoma/fistula clearance opening though said wafer can be custom cut to clear the size of said stoma/fistula or purchased with predetermined stoma/fistula clearance diameter openings.

3. A contained ostomy appliance consisting of an improvement to an ostomy pouch for collecting body wastes/excretions and flagellant gas from a stoma/fistula as in claim 1, wherein said ostomy appliance is a two piece appliance with an adhesive backed attachment wafer that has an attachment flange that snaps into a mating attachment flange that is mounted to said first and second proximal side layers where stoma/fistula clearance openings in said attachment flanges align with said stoma/fistula clearance openings in first and second proximal side layers whereby body wastes/excretions and flagellant gases are collected in said pressure barrier pouch and allowed to escape out said unattached bottom of said pressure barrier pouch into said ostomy pouch.

4. A contained two piece ostomy appliance consisting of an improvement to an ostomy pouch for collecting body wastes/excretions and flagellant gas from a stoma/fistula as in claim 1 wherein said ostomy pouch is assembled around a pre-assembled pressure barrier pouch where said pouches each have their own concentrically mounted attachment flanges and mate with an adhesive backed attachment wafer with a dual acting attachment flange that allows snapping of said attachment flanges of said ostomy pouch and said pre-assembled pressure barrier pouch to said dual acting attachment flange on said adhesive backed attachment wafer, without breaking the seal of the adhesive to the patient's skin.
